(19) United States
(12) Patent Application Publication
(10) Pub. No.: US 2002/0165119 A1
(43) Pub. Date: Nov. 7, 2002

(54) Method of treating inflammatory conditions by inhibiting cytosolic phospholipase A2

(76) Inventor: Alan Leff, Chicago, IL (US)

Correspondence Address: MERCHANT & GOULD PC, P.O. BOX 2903, MINNEAPOLIS, MN, 55402-0903, US

(21) Appl. No.: 10/062,730

(22) Filed: Jan. 31, 2002

Related U.S. Application Data
(60) Provisional application No. 60/265,298, filed on Jan. 31, 2001.

(51) Int. Cl. A61K 31/00; A61K 31/60; A61K 31/415

(52) U.S. Cl. 514/1; 514/406; 514/165

(57) ABSTRACT

Methods for treating or modulating inflammatory processes or chronic inflammatory conditions dependent upon cellular inflammation, such as asthma and rheumatoid arthritis are provided, as well as methods for inhibiting or blocking eosinophil migration and airway hyperresponsiveness. Also described is a method for treating or preventing the adhesion of granulocytes and other inflammatory cells into the tissue that is the site of the inflammation. In particular, the methods relate to the therapeutic or prophylactic use of compounds and compositions that inhibit cytosolic phospholipase A₂.

### FIELD OF THE INVENTION

The present invention relates to methods for treating or modulating inflammatory processes or chronic inflammatory conditions dependent upon cellular inflammation. The invention also relates to the therapeutic or prophylactic use of compounds and compositions that inhibit or reduce the activity of cytosolic phospholipase A₂.

### BACKGROUND OF THE INVENTION

Molecular adhesion is the essential step by which inflammatory cells enter tissues in all diseases. An important regulatory step in this process is adhesion of granulocytes and inflammatory cells to the endothelium, after which these cells migrate by diapedesis into the tissue that is the site of the inflammation.

One common example whereby inflammatory cells enter and migrate into tissues is asthma. Asthma is characterized by acute and chronic inflammation of the airway mucosa that is caused by recruitment of inflammatory cells into the airway parenchyma. There is increasing awareness of the essential role of inflammation of airways with eosinophils in the pathobiology of asthma. Eosinophils are characteristically present within the epithelial layer, and there is accumulation of chronic inflammatory cells such as lymphocytes, plasma cells and macrophages in the lamina propria. Laitinen et al., Am. Rev. Respir. Dis., 147, 697-704 (1993).

The mechanism regulating either the acute or chronic recruitment of inflammatory cells is unknown. Recent studies using cytosolic phospholipase A₂ (cPLA₂) knockout mice have demonstrated diminished airway responsiveness to antigen challenge (Uozumi et al., Nature, 390, 618-622 (1997)) and reduced pulmonary edema in sepsis or acid aspiration (Nagase et al., Nature Immunol., 1, 42-46 (2000)), suggesting a potential regulatory role of cPLA₂ in inflammatory cell migration into airways.

PLA₂ is the rate-limiting enzyme involved in the conversion of membrane phospholipids to arachidonic acid (AA) and lysophospholipids. See, e.g., Hanahan, D. J., Annu. Rev. Biochem., 55, 483-509 (1986). Further catalysis of AA initiates the biosynthesis of potent inflammatory mediators, i.e. prostaglandins (PG)s and leukotrienes (LT)s. Lysophospholipid is converted directly into platelet activating factor (PAF), which is a potent agonist in inducing eosinophil secretion. PAF also has been implicated in the infiltration of eosinophils into asthmatic airways. Barnes, P. J., J. Allergy Clin. Immunol, 83, 1013-1026 (1989). The 5-lipoxygenase (5-LO) product, LTB₄, is a potent chemoattractant for guinea pig and rat eosinophils but is not exceptionally active on human eosinophils in vitro. Powell et al., J. Immunol., 154, 4123-4132 (1995). PAF and AA metabolites, cysteinyl (cys)LTs, (5-LO products), PGD₂, and thromboxane A₂ (TXA₂) (cyclooxygenase [COX] products) also cause bronchoconstriction and airway hyperresponsiveness.

The 85 kDa (Type IV) cPLA₂ is AA-selective and is regulated by physiological intracellular Ca²⁺ concentrations and phosphorylation. Activation of type IV cPLA₂ is thought to be the predominant mechanism by which eicosanoid synthesis is regulated. cPLA₂ may also be an important regulatory enzyme in integrin-adhesion, which is the initial step in eosinophil migration. Zhu et al., J. Immunol., 163, 3423-3429 (1999). These in vitro studies indicated that cPLA₂ is an important messenger protein for maintenance of β₁- and β₂-integrin adhesion to plated ICAM-1 and VCAM-1, respectively.

There is still a need, however, to examine the role of cPLA₂ on antigen-induced eosinophil migration and airway hyperresponsiveness. Moreover, it is desirable to examine whether airway hyperresponsiveness and eosinophil migration caused by cPLA₂ activation resulted from first step synthesis of PAF and lysophospholipids or from downstream metabolism of AA, i.e. COX or 5-LO pathway products.

### SUMMARY OF THE INVENTION

In a general aspect, the invention relates to a method for treating or modulating inflammatory processes or chronic inflammatory conditions dependent upon cellular inflammation, comprising administering to animals or humans, a therapeutically effective amount of a cytosolic phospholipase A₂ (cPLA₂) inhibitor or a pharmaceutically acceptable salt, hydrate, ester, solvate, prodrug, metabolite, or stereoisomer thereof.

In one embodiment, the invention relates to a method for treating or modulating inflammatory processes or chronic inflammatory conditions selected from asthma, rhinitis, idiopathic pulmonary fibrosis, adult respiratory distress syndrome, pulmonary edema associated with sepsis and gastric acid aspiration, rheumatoid arthritis, and inflammatory diseases of the bowel.

In another embodiment, the invention relates to a method for inhibiting, interfering with, or blocking leukocytes (also called white blood cells) migration and pulmonary and nasal airway hyperresponsiveness, comprising administering to an animal or a human a therapeutically effective amount of a cPLA₂ inhibitor or a pharmaceutically acceptable salt, hydrate, ester, solvate, prodrug, metabolite, or stereoisomer thereof.

In another embodiment, the invention relates to a method for inhibiting, interfering with, or blocking inflammatory cells, including white blood cells from adhering to target tissues. As used herein, the terms leukocyte and white blood cells are used interchangeably to refer to cells that circulate in the blood and lymphatic system and harbor in the lymph glands and spleen, which are part of the immune system responsible for attacking foreign invaders of the body. White blood cells can be divided into two main types: Granulocytes, which include basophils, eosinophils and neutrophils; and Agranulocytes, which include monocytes and lymphocytes. Additionally, the terms “white blood cells” and “leukocytes”, as used herein, refers to mast cell precursors, T-helper cells and precursors thereof. The method comprises administering to an animal or a human a therapeutically effective amount of a compound or a pharmaceutically acceptable salt, hydrate, ester, solvate, prodrug, metabolite, or stereoisomer thereof, that inhibits or reduces the activity of platelet activating factor. In one embodiment, the inhibitor is a PAF antagonist such as CV6209. In another embodiment, the compound is one that inhibits or reduces the activity of cPLA₂.

In another embodiment, the invention relates to a composition comprising a cPLA₂ inhibitor or a pharmaceutically acceptable salt, hydrate, ester, solvate, prodrug, metabolite, or stereoisomer thereof; wherein the cPLA₂ inhibitor is present in an amount that is effective for treating or preventing inflammatory processes or chronic inflammatory conditions dependent upon cellular inflammation.

In one embodiment, the cPLA₂ inhibitor is a compound capable of binding to and/or blocking the serine-228 residue, an active catalytic site of cPLA₂, thereby blocking the activity of cPLA₂. Suitable cPLA₂ inhibitors include methylketones, such as trifluoromethylketone (TMFK) or Arachadonic AcidBrMethylKetone(AABrMK), a derivative of TFMK. (Biochemistry 1966, 35: 371-372). Surfactin, a complex lipid, has also been shown to inhibit or reduce the activity of cPLA₂ (Zhu X, Munoz MN et al. J. Immunol. 163:3423-3429, 1999). Additionally, platelet activating factor (PAF) antagonists, and derivatives or metabolites thereof have been shown to inhibit or reduce the activity of cPLA₂.

Another class of compounds that are capable of inhibiting or reducing the activity of cPLA₂ include those capable of interfering with serine-505-phosphorylation of cPLA.

The invention also relates to the inhibition of or reduction in the expression of cPLA₂, for example, by using antisense oligonucleosides. Antisense mRNA can be administered by inhalation, topically or parenterally, by infusion or injection, for local or systemic application for the treatment of allergic inflammatory conditions such as allergic rhinitis, asthma, atopic dermatitis, psoriasis, idiopathic pulmonary fibrosis, adult respiratory distress syndrome, pulmonary edema associated with sepsis and gastric acid aspiration, as well as inflammatory bowel disease and rheumatoid arthritis. In one embodiment, the antisense oligonucleoside is used in combination with another antisense sequences designed to block or modify expression of other cytokines, adhesion molecules or mediators that play a role in the initiation and maintenance of the allergic or non-allergic inflammatory process. Examples of other pro-inflammatory cytokines or mediators include, but are not limited to, cysteinyl leukotrienes, PAF or lysophospholipids, IL⁴, IL⁵, IL¹³, ICAM^{1,2 and 3}, VCAM¹, VLA⁴, IgE, IL⁸ and TNFα.

### BRIEF DESCRIPTION OF THE FIGURES

FIG. 1 demonstrates that immediate bronchoconstriction in guinea pigs after ovalbumin (OA)-challenge was not mediated by metabolites of cPLA₂ hydrolysis. Antigen challenge caused an increase in specific airway resistance (sRaw) in OA-sensitized animals from 1.6±0.1 (negative controls; saline inhalation) to 23.9±2.6 cmH₂O·s (positive controls; OA-inhalation) (p<0.01). The H₁-receptor antagonist, ebastine, significantly attenuated the increase in sRaw, suggesting that the immediate response was caused substantially by mast cell release of histamine (FIG. 1d). However, this increase in sRaw caused by OA challenge was not attenuated by pre-treatment with either trifluoromethyl ketone (TFMK), a selective cPLA₂ inhibitor, E6123, a PAF receptor antagonist, indomethacin, a COX inhibitor, AA-861, a 5-LO inhibitor, or a combination of indomethacin and AA-861 (FIGS. 1a-c) (p=NS vs positive controls).

FIG. 2 is a schematic showing a generic methylketone structure (FIG. 2a), trifluoromethyl ketone (TFMK) (FIG. 2b) and Arachadonic acid bromic methyl ketone (AAbrMK) (FIG. 2c).

FIG. 3 demonstrates that late phase bronchial hyperresponsiveness to methacholine was mediated by cPLA₂ through first step catalysis into PAF. Within 24 h after antigen challenge, sRaw decreased to baseline (pre-challenge). However, the PC₂₀₀ (provocative concentration of methacholine causing a 200% increase in airway opening pressure [Pao]) decreased from 257±0.07 μg/ml for saline-challenged control guinea pigs to 99.0±0.07 μg/ml for OA-challenge animals (p<0.01). In animals pretreated with the cPLA₂ inhibitor TFMK, the decrease in PC₂₀₀ was blocked in dose-dependent manner to 218±0.08 μg/ml for antigen challenged animals pretreated with 20 mg/kg TFMK vs 99.0±0.07 μg/ml for antigen-challenged animals pretreated with saline (p<0.01). The PAF antagonist, E6123, also caused complete blockade of ovalbumin-induced methacholine hyperresponsiveness (p<0.01 vs positive control) (FIG. 3c).

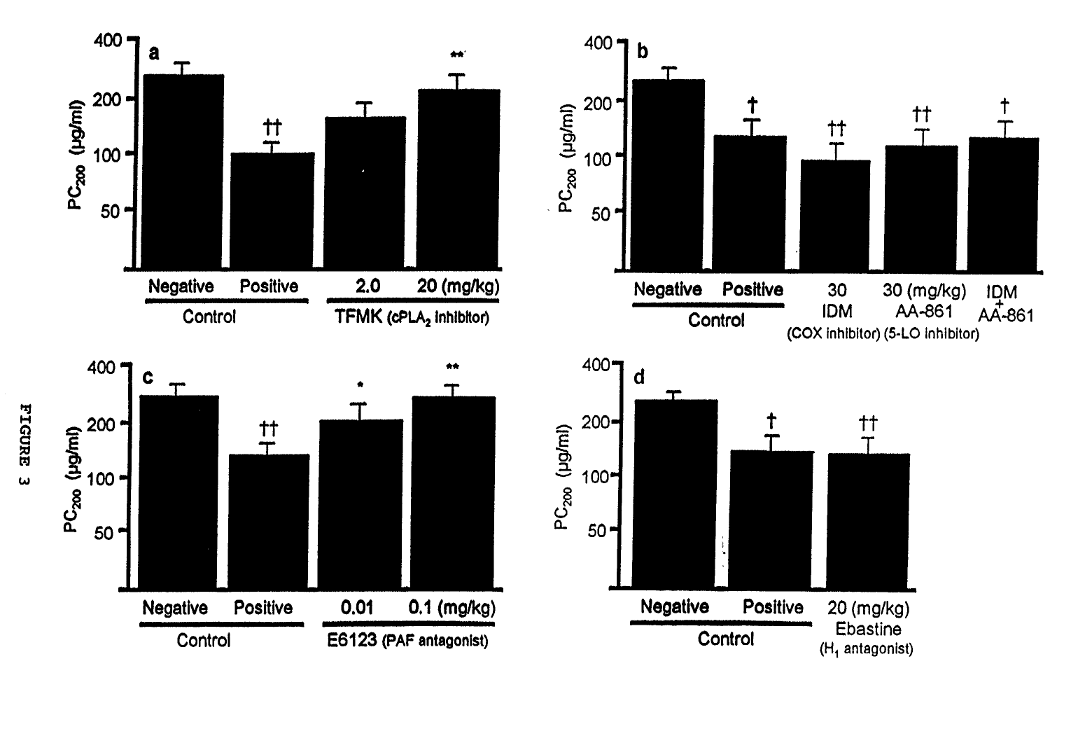

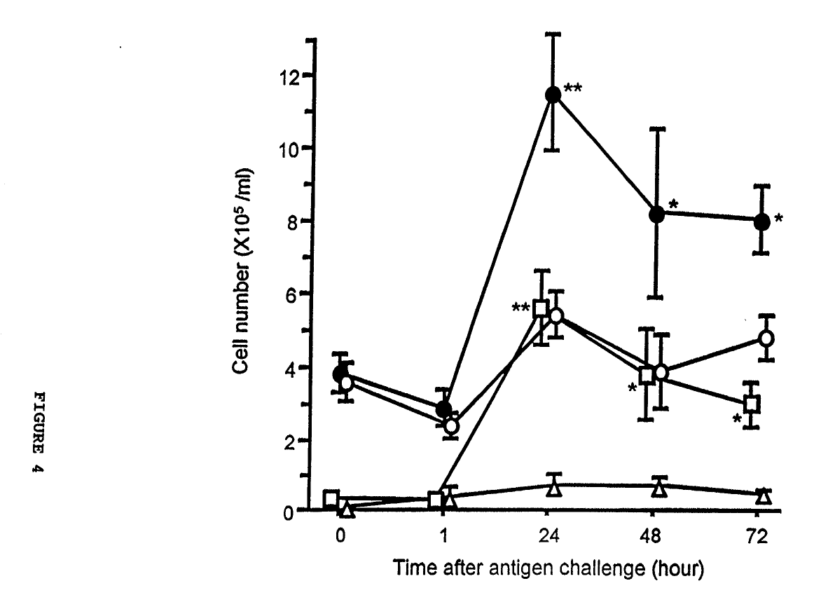

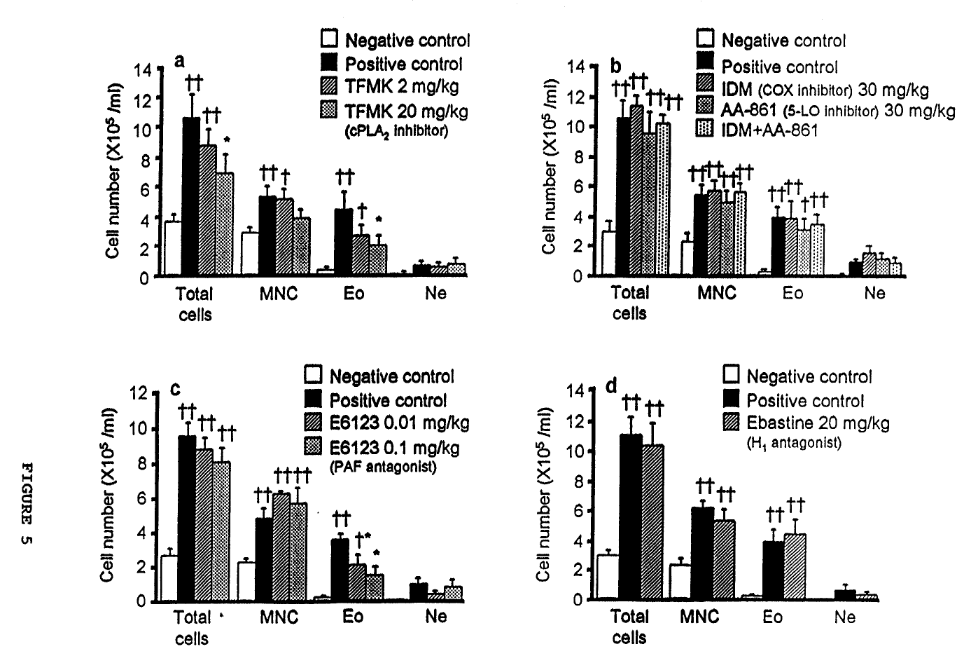

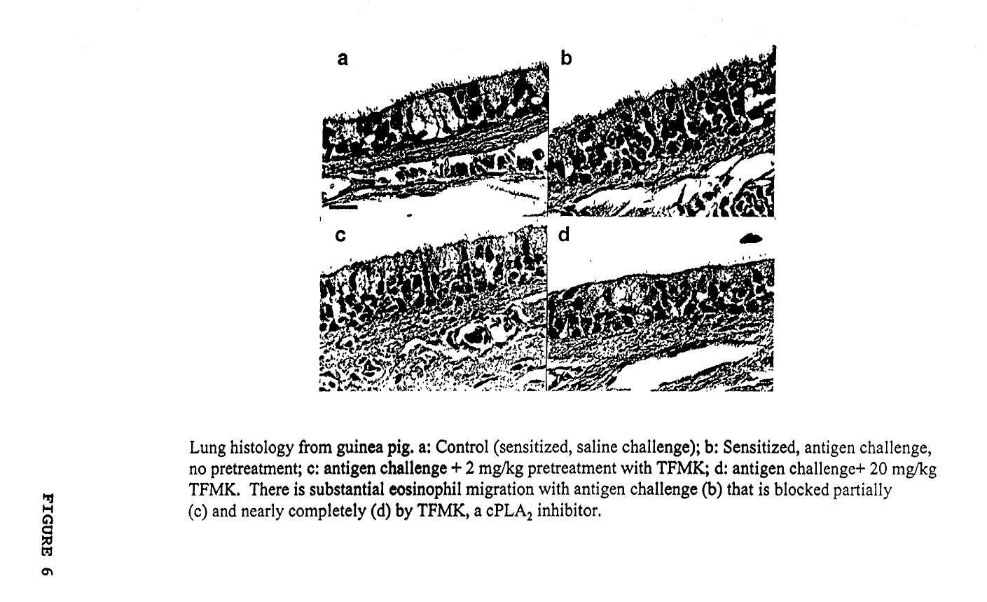

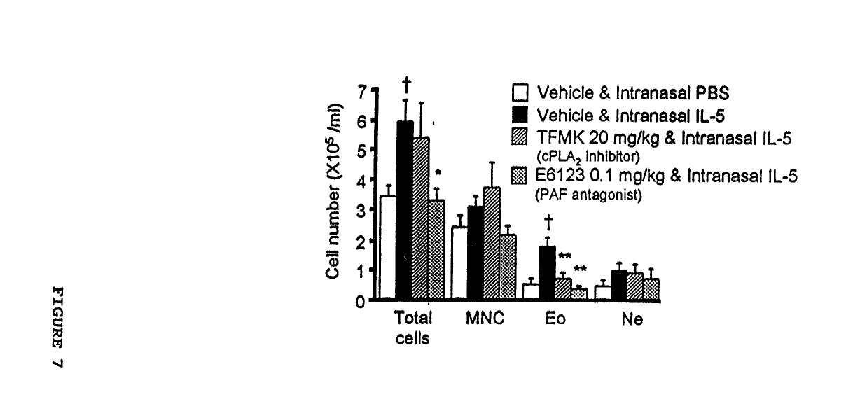

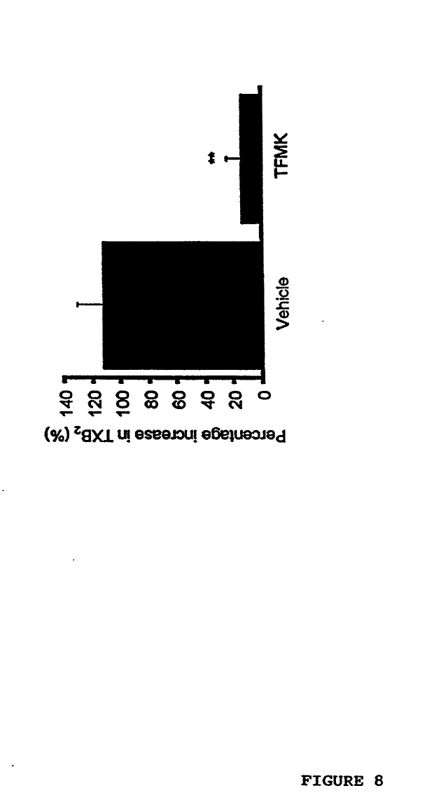

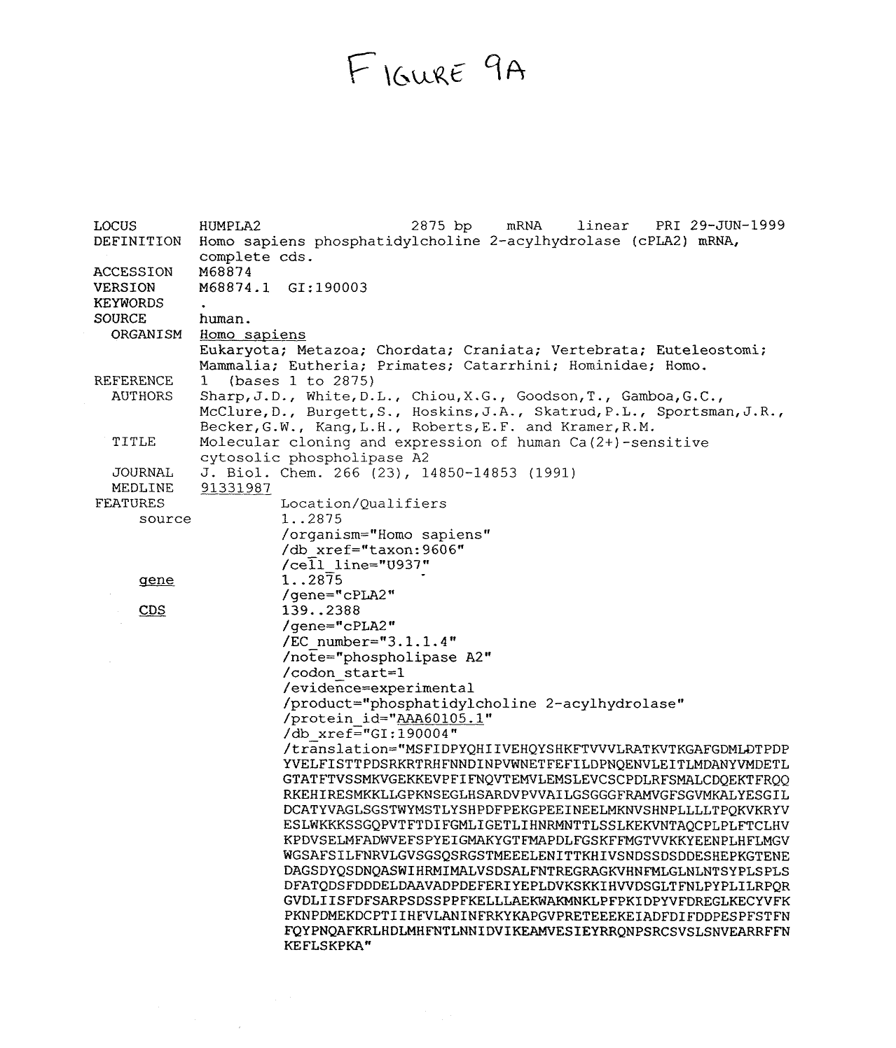

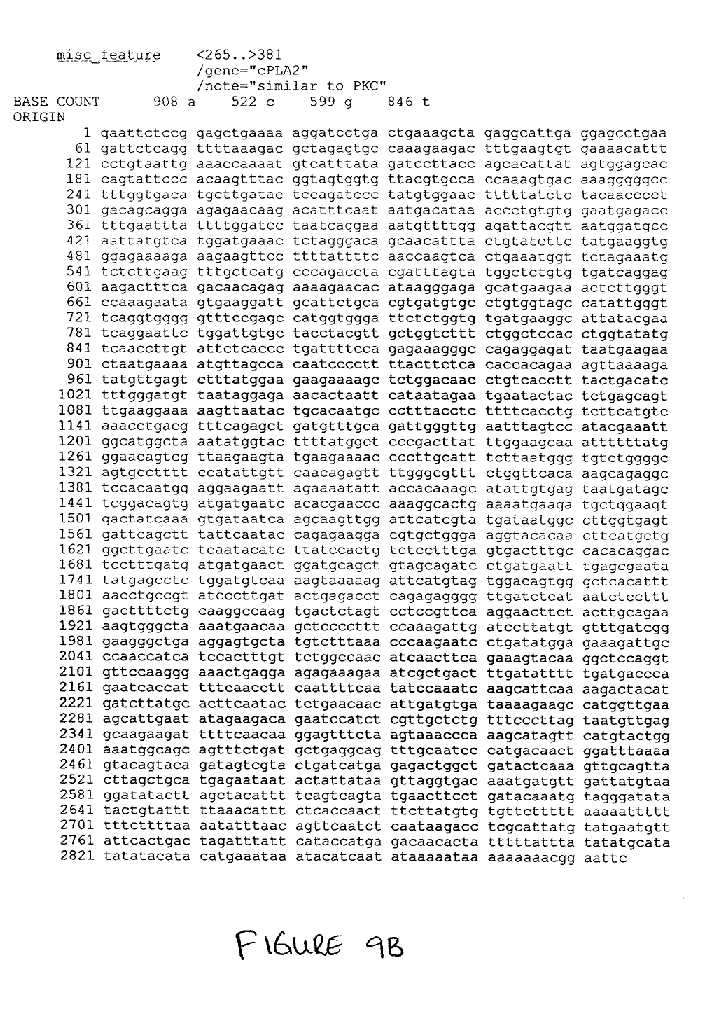

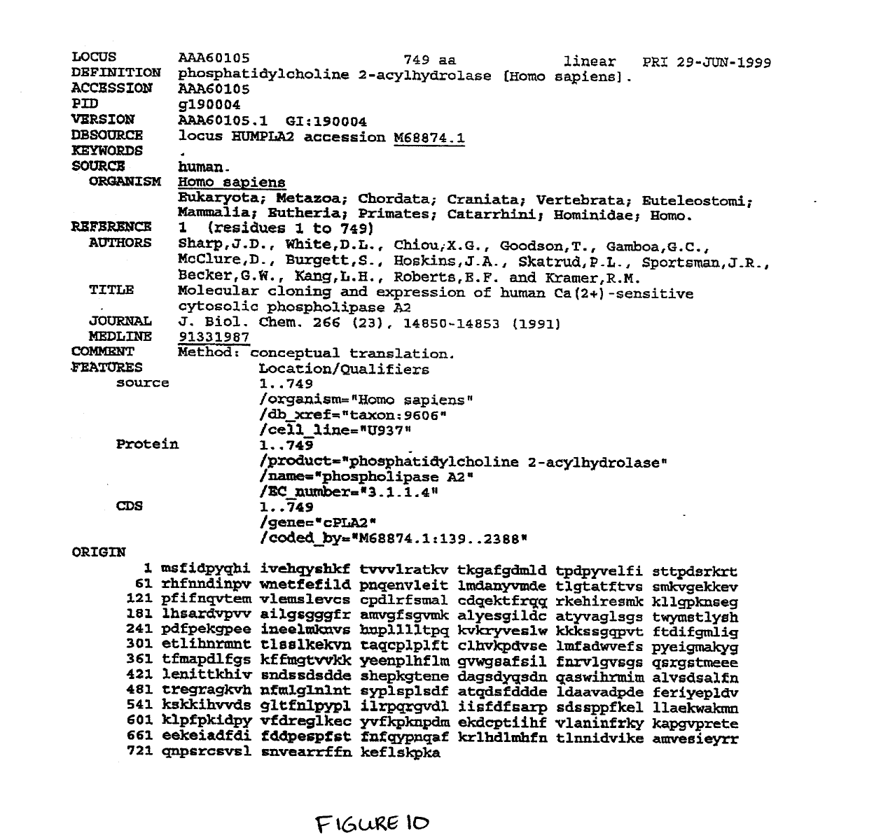

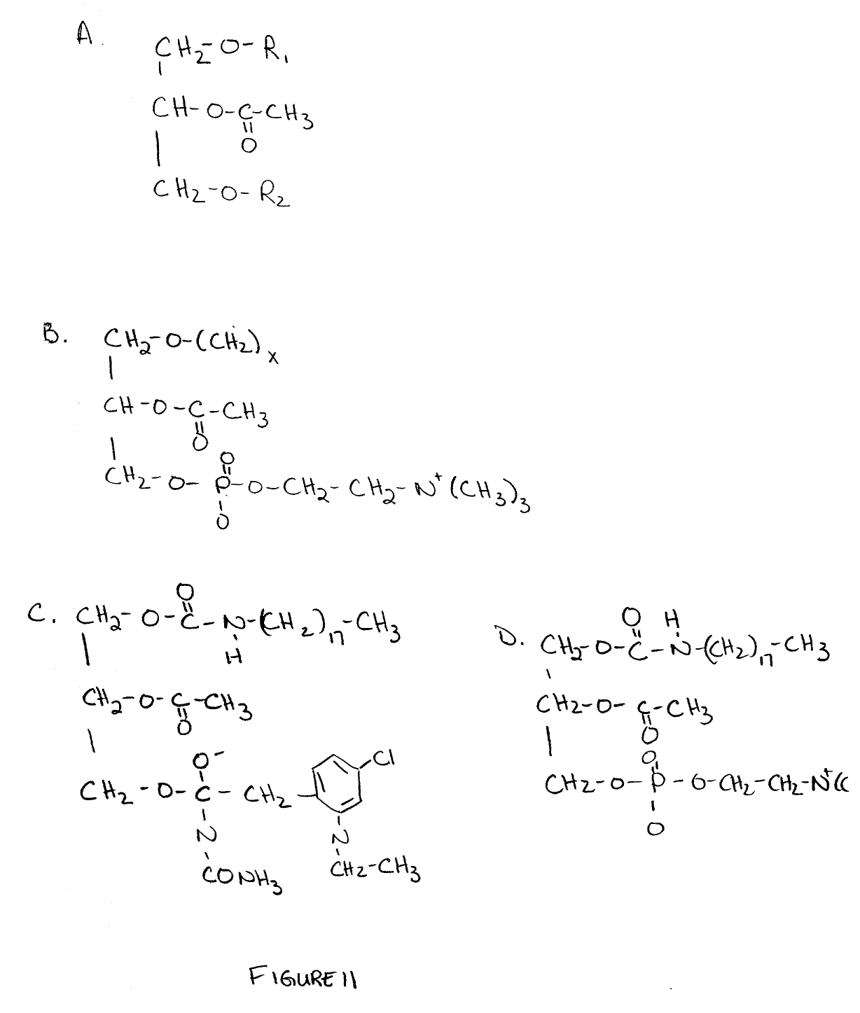

By contrast, neither indomethacin, a COX inhibitor, nor AA861, a 5-LO inhibitor, blocked antigen-induced airway hyperresponsiveness (FIGS. 3b and d). The H₁-receptor antagonist, ebastine, which blocked antigen challenge at 60 min had no effect on OA challenge at 24 h. Blockade with TFMK and E6123 was specific, as baseline airway responsiveness to methacholine was not altered by either TFMK or E6123 alone. Airway hyperresponsiveness was measured at a time when Pao was comparable for all groups. Hence, the measured increase in airway responsiveness did not result from residual bronchomotor tone after methacholine inhalation.

FIG. 4 shows the change in inflammatory cell numbers in the airway lumen after antigen challenge. Bronchoalveolar lavage (BAL) was performed before and after antigen challenge, and total and differential cell counts were obtained. Antigen-inhalation significantly increased total cell numbers from 3.7±0.5×10⁵ before challenge to 11.4±1.6×10⁵/ml at 24 h, and was still increased 72 h after antigen-challenge (p<0.05). Eosinophils in BAL fluid were 0.2±0.1×10⁵/ml before antigen challenge, 5.5±1.0×10⁵/ml at 24 h, and still were increased 72 h after antigen-challenge (p<0.05 vs control at 24 and 72 hr). Mononuclear cells also increased at 24 h and remained increased for 72 h, while neutrophil number was unchanged after antigen challenge.

FIG. 5. Pretreatment of animals with either TFMK or E6123 blocked BAL eosinophilia at 24 h after OA-challenge from 4.5±1.2 and 3.6±0.4×10⁵/ml to 2.1±0.7×10⁵/ml (p<0.05) and 1.6±0.6×10⁵/ml (P<0.05 vs control), respectively (FIGS. 5a and c). However, there was no effect of COX inhibition with indomethacin nor 5-LO inhibition with AA-861 after ovalbumin challenge on eosinophil count in BAL fluid (FIG. 5b). No effect on mononuclear cell or neutrophil count was observed with these inhibitors.

FIG. 6 shows histology after ovalbumin challenge. Histological samples of bronchial airways demonstrated consistently that ovalbumin-challenge induced inflammatory cell infiltration (largely eosinophils) in the epithelial layer and lamina propria (FIG. 6b vs a). Pretreatment with 2-20 mg/kg TFMK blocked cell infiltration progressively at 24 h (FIGS. 6c and d).

FIG. 7. Studies were performed to determine the role of cPLA₂ in eosinophil migration caused by IL-5. See Serhan, C. N., Prostaglandins, 53, 107-137 (1997). IL-5 significantly increased total cell numbers from 3.5±0.3×10⁵ for vehicle treated control animals to 5.9±0.7×10⁵/ml (p<0.05). Pretreatment of animals with the PAF antagonist, E6123, decreased total cell number to 3.3±0.4×10⁵/ml (P<0.05).

BAL eosinophils were significantly increased from 0.6±0.2×10⁵/ml to 1.8±0.3×10⁵/ml 24 h after IL-5 treatment (P<0.05). Pretreatment of animals with either the cPLA₂-inhibitor, TFMK, or the PAF antagonist, E6123, blocked BAL eosinophilia to 0.7±0.2×10⁵ (p<0.01) and 0.4±0.1×10⁵/ml (P<0.01), respectively. Mononuclear cells and neutrophil number did not change 24 h after IL-5 treatment.

FIG. 8. To determine whether the dose of TFMK administered by i.p. injection in vivo was capable of inhibiting eicosanoid production caused by cPLA₂, I examined the effects of TFMK administration on production of TXB₂. N-formyl-methionyl-leucyl-phenylalanine (FMLP) (10⁻⁶ M)+5 μg/ml cytochalasin B (CB) caused a 111±18.6% increase in stimulated TXB₂ production from whole blood, which was significantly inhibited to a 13.5±10.5% increase after pretreatment with 20 mg/kg i.p. TFMK (p<0.01 vs vehicle).

FIG. 9 shows the nucleotide sequence for cPLA₂.

FIG. 10 shows the amino acid sequence for cPLA₂.

FIG. 11 is a schematic showing various PAF inhibitors.

### DETAILED DESCRIPTION OF THE INVENTION

The terms “comprising” and “including” are used herein in their open, non-limiting sense.

The terms “treat”, “treatment” and “therapy” refers to curative therapy, prophylactic therapy and preventative therapy, and can mean:

(i) preventing a disease, disorder, or condition from occurring in an animal or humans that may be predisposed to the disease, disorder and/or condition, but has not yet been diagnosed as having it;

(ii) arresting or slowing the development of a disease; and

(iii) relieving the disease, disorder, or condition, i.e., causing regression of the disease, disorder, and/or condition.

“A pharmaceutically acceptable prodrug” is a compound that may be converted under physiological conditions or by solvolysis to the specified compound or to a pharmaceutically acceptable salt of such compound prior to exhibiting its pharmacological effect (s). Typically, the prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the cPLA₂ inhibitors using methods known in the art, such as those described by Burger's Medicinal Chemistry and Drug Chemistry, 1, 172-178, 949-982 (1995). See also Bertolini et al., J. Med. Chem., 40, 2011-2016 (1997); Shan, et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); and Larsen, Design and Application of Prodrugs, Drug Design and Development (Krogsgaard-Larsen et al., eds., Harwood Academic Publishers, 1991).

“A pharmaceutically active metabolite” is intended to mean a pharmacologically active product produced through metabolism in the body of a specified compound or salt thereof. After entry into the body, most drugs are substrates for chemical reactions that may change their physical properties and biologic effects. These metabolic conversions, which usually affect the polarity of the cPLA₂ inhibitor, alter the way in which drugs are distributed in and excreted from the body. However, in some cases, metabolism of a drug is required for therapeutic effect. For example, anticancer drugs of the anti-metabolite class must be converted to their active forms after they have been transported into a cancer cell.

Since most drugs undergo metabolic transformation of some kind, the biochemical reactions that play a role in drug metabolism may be numerous and diverse. The main site of drug metabolism is the liver, although other tissues may also participate.

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, famarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

Role of Cytosolic Phospholipase A₂

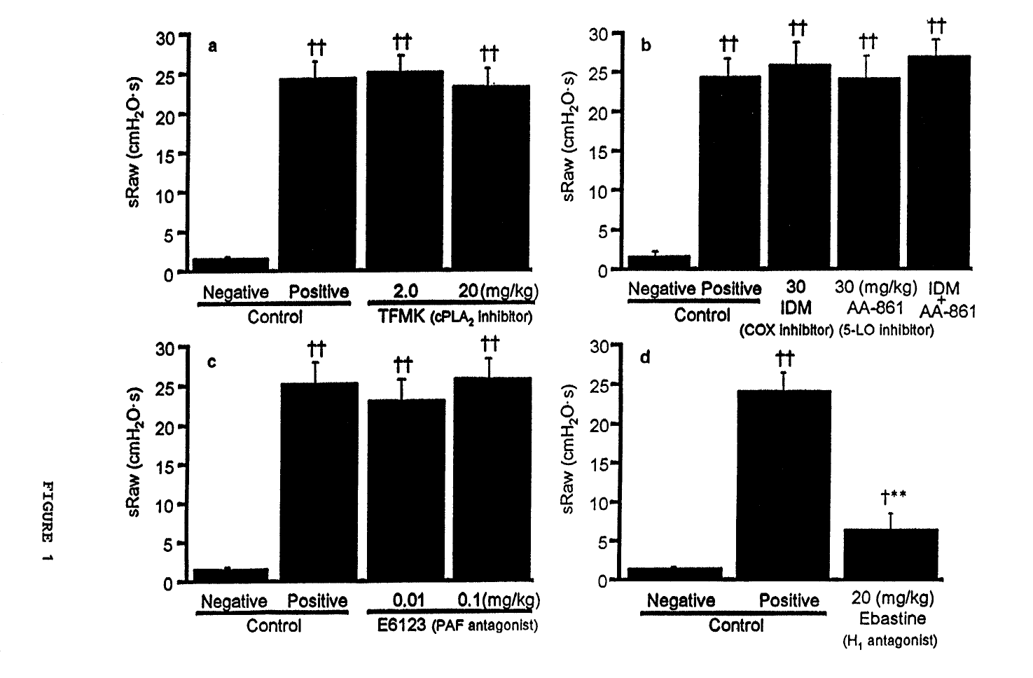

This invention relates to the role of cPLA₂ on antigen-induced early phase bronchoconstriction and on subsequent late phase airway inflammation and airway hyperresponsiveness after antigen inhalation. The inventors have discovered that trifluroromethylketone (TFMK) causes a dose-dependent attenuation of both antigen-induced airway eosinophil accumulation and airway hyperresponsiveness to methacholine. These effects occurred even though the immediate response to antigen was not blocked. These findings suggest that cPLA₂ is involved in antigen-induced late phase airway reactions and that early phase bronchoconstriction is caused largely or completely by anaphylactic release of histamine in guinea pig airways. Notably, HI-antagonism caused nearly complete blockade of antigen challenge (FIG. 1d), but had no substantial effect on methacholine challenge at 24 h (FIG. 3d).

Using the cyclooxygenase (COX) inhibitor, indomethacin, and the 5-liposygenase (5-LO) inhibitor, AA-861, it appears that neither COX nor 5-LO metabolites of arachidonic acid (AA) are involved in antigen-induced airway constriction (FIGS. 3 and 5). By contrast, the PAF receptor antagonist, E6123 (see Tsunoda et al., Agents Actions Suppl, 31, 251-254 (1990)), inhibited both antigen-induced bronchial hyperresponsiveness and airway eosinophil accumulation (FIGS. 3c and 5). Thus, it appears that cPLA₂-catalysis of phospholipid may mediate the late-phase airway reaction.

Additionally, ex vivo FormolMetLeu Phe/cytochalasinB(FMLP/CB)-induced thromboxane B₂ (TXB₂) release from whole blood was inhibited at the doses of TFMK used in these studies (FIG. 8) showing that the hydrolysis of cell membranes or lipid bodies is blocked by inhibiting CPLA2 activity. Prior investigations confirm that the doses of E6123, a PAF receptor antagonist commercially available from Eisaico, Ltd (Tokyo, Japan) (Kaneko et al., Eur. J. Pharmacol., 292, 251-255 (1995), AA-861 (Ashida et al., Prostaglandins, 26, 955-972 (1983), and indomethacin (Arima et al., Chest, 108, 529-534 (1995) are selective and specific for receptor or enzyme blockade in guinea pigs. The effect of TFMK and E6123 appears to be specific to antigen-induced airway hyperresponsiveness, since neither drug alone had any effect on sRaw or baseline airway responsiveness. Therefore, the effects of TFMK and E6123 on antigen-induced airway hyperresponsiveness cannot be attributed to anticholinergic or bronchodilatory effects of the compound.

Airway eosinophil influx after antigen challenge or treatment with interleukin-5 (IL-5) was dose-dependently blocked by the cPLA₂-inhibitor, TFMK, and E6123. This finding suggests that cPLA₂ may act to promote eosinophil migration and subsequent airway hyperresponsiveness through its ability to synthesize lysophospholipid (e.g., PAF) in the first step of membrane lipid hydrolysis.

Thus, it appears that cPLA₂ hydrolysis of phospholipids into lysophospholipid may regulate both granulocyte and eosinophil migration as well as airway hyperresponsiveness to antigen challenge. Accordingly, cPLA₂ may be an important enzyme in the regulation of granulocyte and eosinophil adhesion and migration, and airway hyperresponsiveness. Blockade of either cPLA₂ or its first step metabolite, PAF, causes nearly complete attenuation of eosinophil migration and of airway hyperresponsiveness 24 h after treatment. Blockade of metabolites of AA does not affect either cell migration or airway hyperresponsiveness, demonstrating that these effects are not related to synthesis of eicosanoids. Additionally, expression of chronic airway responsiveness during antigen challenge appears to be time-dependent and early responsiveness likely represents a local anaphylactic response that largely is mediated by histamine. While the data presented here is derived from a guinea pig model of chronic airway inflammation, the findings establish that administration of a small molecular weight compound targeting a single enzyme may be capable of blocking both cell migration and airway hyperresponsiveness. The invention is thus applicable to inflammatory processes of a similar nature that occur spontaneously in humans.

It appears that cPLA₂ has an important role in initiating and maintaining integrin-mediated adhesion of white blood cells and other inflammatory cells to the inflamed or target tissues. Cellular hydrolysis products of cPLA₂ are arachidonic acid and various lysophospholipids. Blockade of lysophospholipid binding and function within inflammatory cells prevents integrin-mediated adhesion of inflammatory cells to target tissues. Examples of drugs that prevent binding and inhibit the lysophospholipid-mediated adhesion are antagonists of platelet activating factor (PAF). The structure for PAF is shown in FIG. 11b. Generally, suitable antagonists of platelet activation factor (PAF) include compounds having a glycerol backbone (see, FIG. 11a) wherein R₁ and R₂ are each independently an alkyl or a heteroalkyl that can be saturated or unstaturated, linear, branched, aromatic and/or cyclic. In one embodiment, R₂ is a polar amine (primary, secondary and/or tertiary). Most typically, R₂ terminates in a secondary or tertiary amine. Examples of such PAF inhibitors include CV6209 (shown in FIG. 11C), or CV3988 (shown in FIG. 11D). Additionally, drugs or chemical compounds that inhibit or reduce the activity of cPLA₂ can be used. Use of these compounds, their pharmaceutically acceptable salts, hydrates, esters, solvates, prodrugs, metabolites, or stereoisomers thereof, or other compounds directed at the targets elucidated above will modulate or lead to complete control of inflammatory processes including asthma, rheumatoid arthritis, inflammatory diseases of the bowel and any other chronic inflammatory conditions dependent upon cellular inflammation.

Inhibitors of Cytosolic Phospholipase A₂

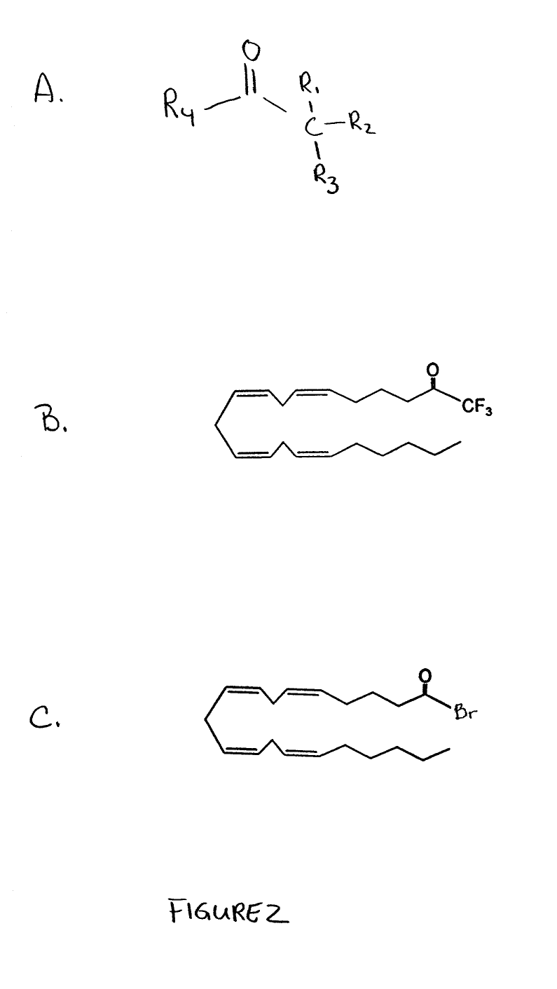

In one embodiment, the cPLA₂ inhibitor is a compound capable of binding to and/or blocking serine-228, the active catalytic site of cPLA₂, thereby blocking the activity of cPLA₂. Suitable inhibitors include methylketones having a structure similar to that of arachadonic acid (CH₃(CH₂)₃(CH₂CH═CH)₄(CH₂)₃CO₂H). Suitable methylketones (See FIG. 2a) include those in which R₁, R₂, and R₃ are each independently hydrogen or halogen and R₄ is a linear C₁₃-C₂₉ alkenyl having between 2 to 8 double bonds. As used herein, the term halogen refers to any of the chemically active elements found in group VIIa of the periodic table; the name applies especially to fluorine (symbol F), chlorine (Cl), bromine (Br), and iodine (I). Chemically they closely resemble one another; they are nonmetallic and form monovalent negative ions. Examples of suitable methylketones include trifluoromethylketone (TMFK) (FIG. 2b) or Arachadonic Acid Bromic Methylketone(AABrMK) (FIG. 2c), a derivative of TFMK. (Biochemistry 1966, 35: 371 372). Additionally, other suitable compounds can be derived from the methylketones mentioned above or those having a similar chemical structure that interacts with the serine-228 active site.

Surfactin (Zhu et al., (1999) J. Immunol. 163:3423-3429) is a complex lipid that has also been shown to inhibit or reduce the activity cPLA₂. Thus, compounds or derivatives of this chemical class, particularly those that prevent the hydrolysis of phospholipid to PAF (as caused by cPLA₂) may also be suitable for use in the method of the invention.

Platelet activating factor (PAF) antagonists and their derivatives, lysophospholipids and other lyso forms and derivatives of PAF or metabolites are also capable of inhibiting or reducing the activity of cPLA₂.

Additionally, compounds that interfere with serine-505-phosphorylation can also be used to inhibit or reduce the activity of cPLA2 because adhesion caused by cPLA2 occurs only in its 505 phosphorylated state.

Therapeutic Use

The invention provides a method of treatment for inflammatory processes or chronic inflammatory conditions. In one embodiment, the invention provides an inhibitor of cPLA2 for use in human as well as veterinary applications. According to the invention, a cPLA2 inhibitor is used therapeutically to suppress or inhibit inflammation, such as that associated with allergic rhinitis, asthma, atopic dermatitis, psoriasis, idiopathic pulmonary fibrosis, adult respiratory distress syndrome, pulmonary edema associated with sepsis and gastric acid aspiration, as well as other inflammatory diseases including inflammatory bowel disease and rheumatoid arthritis. For such purposes, the inhibitor is employed in pharmaceutical compositions, containing one or more active ingredients plus one or more pharmaceutically acceptable carriers, diluents, fillers, binders and other excipients, depending upon the mode of administration and dosage form contemplated.

Pharmaceutical Composition

Another aspect of the invention is directed towards a pharmaceutical composition. The pharmaceutical composition includes an effective amount of cPLA₂ inhibitor, or a pharmaceutically acceptable salt, hydrate, ester, solvate, prodrug, metabolite, or stereoisomer thereof, and a pharmaceutically acceptable excipient or carrier. Suitable pharmaceutical carriers are known. Typically, an appropriate amount of a pharmaceutically acceptable salt is used in the formulation to render the formulation isotonic. Examples of the carrier include buffers such as saline, Ringer's solution and dextrose solution. The pH of the solution is preferably from about 5 to about 8, and more preferably from about 7.4 to about 7.8. Further carriers include sustained release preparations such as semipermeable matrices of solid hydrophobic polymers, which matrices are in the form of shaped articles, e.g., films, liposomes, or microparticles. It will be apparent to those persons skilled in the art that certain carriers may be more preferable depending upon, for instance, the route of administration and concentration of the compound being administered.

The pharmaceutical composition of the present invention is used in amounts that are therapeutically effective and the amounts used may depend upon the desired release profile, the concentration of the pharmaceutical composition required for the desired effect, and the length of time that the pharmaceutical composition has to be released for treatment.

The route of administration is in accord with known methods, e.g., by injection or infusion by intravenous, intramuscular, intracerebral, intraperitoneal, intracerobrospinal, subcutaneous, parenteral, intraocular, intraarticular, intrasynovial, intrathecal, oral, topical, or inhalation routes, or by sustained-release systems. Preferred pharmaceutical compositions include those that can be administered orally to the gastrointestinal tract, parenterally by injection or by inhaler devices known to those in art, e.g., a metered-dose inhaler (MDT), dry powder inhaler (DPI), or nebulizer. The purpose of MDI, (DPI) and a nebulizer is to deposit a clinically effective amount of active compound in the lungs of the patient. By “clinically effective amount of active compound” is meant that amount of active compound that is required to elicit the desired clinical response.

The inhibitor may be delivered to the patient by known methods. Preferably, the inhibitor is mixed with a delivery vehicle and administered by inhalation. The composition typically contains a pharmaceutically acceptable carrier mixed with the agent and other components in the pharmaceutical composition. By “pharmaceutically acceptable carrier” is intended a carrier that is conventionally used in the art to facilitate the storage, administration, and/or the healing effect of the agent. A carrier may also reduce any undesirable side effects of the agent. A suitable carrier should be stable, i.e., incapable of reacting with other ingredients in the formulation. It should not produce significant local or systemic adverse effect in recipients at the dosages and concentrations employed for treatment. Such carriers are generally known in the art.

Acceptable carriers, excipients, or stabilizers are preferably nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

The inhibitor can also be administered in the form of sustained-released preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid (EP 133,988).

The therapeutically effective dose will, of course, vary depending on such factors as the intended therapy (e.g., for modulating inflammatory responses), the pathological condition to be treated, the method of administration, the type of compound being used for treatment, any co-therapy involved, the patient's age, weight, general medical condition, medical history, etc., and its determination is well within the skill of a practicing physician. Accordingly, it will be necessary for the therapist to titer the dosage and modify the route of administration as required to obtain the maximal therapeutic effect.

Antisense Technology

The invention also relates to the inhibition of the expression of cPLA₂ by treatments with antisense oligonucleosides targeting disease specific mRNA. The invention also relates to the use antisense oligonucleosides directed at preventing the expression of cPLA₂ as specified above, that are also combined with specific other antisense sequences that are designed to block or modify other disease specific mRNAs that result in the expression of any of the other cytokines, adhesion molecules or mediators that play a role in the initiation and maintenance of the allergic or non-allergic inflammatory process. Examples of other cytokines, adhesion molecules or mediators include, but are not limited to, cysteinyl leukotrienes, PAF or lysophospholipids are, IL⁴ (Interleukin⁴), IL⁵ (Interleukin⁵), IL¹³ (Interleukin¹³) ICAM^{1,2 and 3} (Intercellular Adhesion Molecule^{1,2,3}), VCAM¹ (Vascular Cell Adhesion Molecule¹), VLA⁴, IgE (Immunoglobulin E), IL⁸ (Interleukin⁸) and TNFα (Tumor Necrosis Factor Alpha). This invention therefore in general aspect also relates to combination therapy with anti-sense compounds where one of the targets is cPLA₂ and any of the other targets is one or more of the pro-inflammatory cytokines or mediators mentioned above.

Antisense technology entails the administration of exogenous oligonucleotides that bind to a target polynucleotide located within the cells. The term “antisense” refers to the fact that such oligonucleotides are complementary to their intracellular targets. (See for example, Jack Cohen, OLIGODEOXYNUCLEOTIDES, Antisense Inhibitors of Gene Expression, CRC Press, 1989; and Synthesis 1:1-5 (1988)). The antisense oligonucleotides of the invention may be RNA or DNA which is complementary to and stably hybridizes with the cPLA₂ genome or the corresponding mRNA. The nucleic acid sequence for cPLA₂ is shown in FIGS. 9a and 9b. The amino acid sequence is shown in FIG. 10. It is believed that the serine residues at positions 228, 505 and 727 are important for cPLA₂ activity. Therefore, it may be desirable to construct an antisense oligonucleotide that binds to at least one of these residues. For example, an antisense oligonucleotide capable of hybridizing with the nucleotide sequence for cPLA₂ between residues 200 and 800 may be desirable. While absolute complementarity is not required, high degrees of complementarity (e.g., above about 85% complementarity) are preferred. Use of a complementary oligonucleotide allows for the selective hybridization to cPLA₂ mRNA and not to mRNA. Preferably, the antisense oligonucleotides are a 10 to 50-mer (more typically, a 15 to 30-mer) fragment of the antisense DNA molecule having a sequence that hybridizes to mRNA.

If desired, the antisense oligonucleotide can be co-administered with an agent that enhances the uptake of the antisense molecule by the cells, for example, a lipophilic cationic compound that may be in the form of liposomes. The use of liposomes to introduce nucleotides into cells is taught, for example, in U.S. Pat. Nos. 4,897,355 and 4,394,448, the disclosures of which are incorporated by reference in their entirety. See also U.S. Pat. Nos. 4,235,871, 4,231,877, 4,224,179, 4,753,788, 4,673,567, 4,247,411, and 4,814,270 for general methods of preparing liposomes comprising biological materials. Alternatively, the antisense oligonucleotide may be combined with a lipophilic carrier such as any one of a number of sterols including cholesterol, cholate, and deoxycholic acid.

The antisense oligonucleotides of the present invention may be prepared according to any of the methods that are well known to those of ordinary skill in the art. Preferably, the antisense 10 oligonucleotides are prepared by solid phase synthesis. (See Goodchild, J., Bioconjugate Chemistry, 1:165-167 (1990)), for a review of the chemical synthesis of oligonucleotides. Alternatively, the antisense oligonucleotides can be obtained from a number of companies that specialize in the custom synthesis of oligonucleotides.

Anti-cPLA₂ Antibodies

The invention further provides anti-cPLA₂ antibodies. Exemplary antibodies include polyclonal, monoclonal, humanized, bispecific, and heteroconjugate antibodies.

1. Polyclonal Antibodies

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the cPLA₂ polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). Further, polyclonal antibodies may be generated commercially, for example by Genemed Synthesis, Inc. using art-accepted methods.

2. Monoclonal Antibodies

Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the cPLA₂ polypeptide or a fusion protein thereof. Generally, either peripheral blood lymphocytes (“PBLs”) are used if cells of human origin are desired, or spleen cells or lymph node cells are used if nonhuman mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which are substances that can prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J., Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against cPLA₂. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640 medium. Alternatively, the hybridoma cells may be grown in vivo as ascites in a mammal. The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography. The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or mycloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a nonimmunoglobulin polypeptide. Such a nonimmunoglobulin polypeptide can be substituted for the constant domains of an antibody of the invention or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking.

Alternatively, the relevant cysteine residues are substituted with another amino acid residue or are deleted so as to prevent crosslinking. In vitro methods are also suitable for preparing monovalent antibodies. Digestion of antibodies to produce fragments thereof, particularly, Fab fragments, can be accomplished using routine techniques known in the art.

3. Humanized Antibodies

The antibodies may further comprise humanized antibodies or human antibodies. Humanized forms of nonhuman (e.g., murine) antibodies are chimeric immunoglobulins, immunoglobulin chains, or fragments thereof (such as Fv, Fab, Fab′, F(ab′)₂, or other antigen-binding subsequences of antibodies) that contain minimal sequence derived from nonhuman immunoglobulin. Humanized antibodies include human immunoglobulins (recipient antibody) in which residues from a complementary-determining region (CDR) of the recipient are replaced by residues from a CDR of a nonhuman species (donor antibody) such as mouse, rat, or rabbit having the desired specificity, affinity, and capacity. In some instances, Fv framework residues of the human immunoglobulin are replaced by corresponding nonhuman residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a nonhuman immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Reichmann et al., Nature, 332:323-329 (1988); and Presta, Curr. Op. Struct. Biol., 2:593-596 (1992)).

Methods for humanizing nonhuman antibodies are well known in the art. Generally, a humanized antibody has one or more amino acid residues introduced into it from a source that is nonhuman. These nonhuman amino acid residues are often referred to as “import” residues, which are typically taken from an “import” variable domain. Humanization can be essentially performed following the method of Winter and coworkers (Jones et al., Nature, 321:522-525 (1986); Reichmann et al., Nature, 332:323-327 (1988); Verhoeven et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a nonhuman species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol., 227:381 (1991); Marks et al., J. Mol. Biol., 222:581 (1991)). The techniques of Cole et al. and Boemer et al. are also available for the preparation of human monoclonal antibodies (Cole et al., Monoclonal Antibodies and Cancer Therapy, Alan R. Liss, p. 77 (1985) and Boemer et al., J. Immunol., 147(1):86-95 (1991)).

Humanized antibodies can also be prepared according to the methods disclosed by, for is example, U.S. Pat. Nos. 5,175,384; 5,434,340; 5,545,806; 5,569,825; 5,591,669; 5,625,126; 5,633,425; 5,916,771; and 5,589,369.

4. Bispecific Antibodies

Bispecific antibodies are monoclonal, preferably human or humanized, antibodies that have binding specificities for at least two different antigens. In the present case, one of the binding specificities is for cPLA₂, the other one is for any other antigen, and preferably for a cell-surface protein or receptor or receptor subunit. Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors and are cotransfected into a suitable host organism. For further details of generating bispecific antibodies see, for example, Suresh et al., Methods in Enzymology, 121:210 (1986).

F. Uses for Antibodies

The antibodies of the invention have various utilities. For example, antibodies may be used in diagnostic assays. Various diagnostic assay techniques known in the art may be used, such as competitive binding assays, direct or indirect sandwich assays, and immunoprecipitation assays conducted in either heterogeneous or homogeneous phases (Zola, Monoclonal Antibodies: A Manual of Techniques, CRC Press, Inc. (1987) pp. 147-158). The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982). In addition, the antibodies may be used in drug screening assays to identify compounds that act to positively or negatively modulate the function of cPLA₂.

The antibodies can also be cPLA₂ antagonists or agonists. Antibodies may also be useful therapeutically either alone, as agents that would act directly to interfere with the function of cPLA₂ or indirectly as targeting agents capable of delivering a toxin, for example, pseudomonas exotoxin or radioisotopes, conjugated thereto to a desired site.

The following examples are offered for illustrative purposes only and are not intended to limit the scope of the present invention in any way. All patent and literature references cited in the present specification are hereby incorporated by reference in their entirety.

### WORKING EXAMPLES

### Methods and Materials

Chemicals

The following chemicals were used: arachidonic trifluoromethyl ketone (TFMK) (FIG. 2) (Biomol, Plymouth Meeting, Pa.), indomethacin (Wako Pure Chemical Ind., Osaka, Japan), AA-861 (Wako Pure Chemical Ind., Osaka, Japan) recombinant human interleukin-5 (IL-5) (R&D 10 Systems, Inc., Minneapolis, Minn., USA), ovalbumin (Sigma, St. Louis Mo., USA), aluminum hydroxide [Al(OH)₃] (Wako Pure Chemical Ind., Osaka, Japan), diethyl ether (Wako Pure Chemical Ind., Osaka, Japan), sodium pentobarbitone (Abbot Laboratories, North Chicago, U.S.A.), and methacholine (Wako Pure Chemical Ind., Osaka, Japan). E6123 and ebastine were provided by Eisai Co. Ltd. (Tokyo, Japan) and Dainippon Pharmaceutical Ltd. (Osaka, Japan), respectively.

Sensitization of Animals

Male albino Hartley strain guinea pigs were obtained from Sankyou Laboratory Service (Toyama, Japan), and were quarantined in the Animal Research Center of Kanazawa University for 1 wk before study. All animal procedures in this study complied with the standards specified in the Guideline for the Care and Use of Laboratory Animals at the Takara-machi Campus of Kanazawa University.

Guinea pigs weighing 200 to 250 g were actively sensitized by a modification of the method reported by Andersson et al., Int. Arch. Allergy Appl. Immunol., 64, 249-258 (1981). Guinea pigs were pretreated with an i.p. injection of 30 mg/kg of cyclophosphamide. Two days later, the animals were immunized with 2.0 mg of ovalbumin and 100 mg of aluminum hydroxide [Al(OH)₃]. A booster injection of 10 μg of OA together with 100 mg of Al(OH)₃ was given 3 wk after the primary immunization.

Antigen-Induced Bronchoconstriction TFMK, a cPLA₂ inhibitor (2.0 or 20 mg/kg i.p.; n=7 in each group), E6123 (a PAF receptor antagonist, 0.01 or 0.1 mg/kg, i.p.; n=7), indomethacin (a COX inhibitor, 30 mg/kg, i.p., n=5), AA-861 (Yoshimoto et al., Biochim. Biophys. Acta, 713, 470-473 (1982) (a 5-LO inhibitor, 30 mg/kg, i.p., n=5), a combination of indomethacin and AA-861 (n=5), or ebastine (a selective histamine H₁ receptor antagonist, 20 mg/kg, i.p., n=6) was administered 60 min before antigen challenge. Additional animals were studied in control groups receiving the i.p. administered vehicle and either inhalation of saline instead of OA (negative controls) or inhalation of OA (positive controls).

Animals were placed in a whole-body, double-chamber plethysmograph for measurement of specific airway resistance (sRaw) by a modification of the method described by Pennock et al., Appl. Physiol., 46, 399-406 (1996). A respiratory mechanics analyzer (model PMUA+SAR, Buxco Electronics, Troy, N.Y.) was used to measure the phase shift between nasal and thoracic airflow and to compute sRaw on a breath by breath basis. A bias flow of air (20 ml/sec) was maintained through the nasal chamber to ensure a constant supply of fresh air to the animal. The analyzer subtracted this flow from its calculations. Sixty min after i.p. administration of TFMK, E6123, indomethacin, AA-861, or ebastine, animals were challenged by 60 sec exposure to an aerosol of ovalbumin generated from a 10 mg/ml solution in saline by a DeVilbiss 646 nebulizer (DeVilbiss Co., Somerset, Pa., USA) operated by compressed air passed through the nasal chamber at 5 /min. The nebulizer output was 0.14 ml/min. Baseline measurement of sRaw was obtained in all groups 5 min before antigen challenge.

Antigen-Induced Airway Hyperresponsiveness to Methacholine

Antigen-induced airway response to methacholine was measured 24 h after challenge. Guinea pigs were anaesthetized by an i.p. injection of 75 mg/kg of sodium pentobarbitone and were placed in a supine position. After the trachea was cannulated with a polyethylene tube (outside diameter, 2.5 mm; inside diameter, 2.1 mm), the animals were ventilated artificially using a small-animal respirator (Model 1680, Harvard Apparatus Co., Inc., South Natick, Mass.) adjusted to deliver a tidal volume of 10 ml/kg at a rate of 60 strokes/min. Changes in lung resistance to inflation, specifically the lateral pressure of the tracheal tube (pressure at the airway opening; Pao, cmH₂O) were measured using a pressure transducer (TP-603T, Nihon Koden Kogyo Co., Ltd., Tokyo, Japan) according to the modification of the method of Konzett and Roessler et al., Arch. Exp. Path. Pharmak., 195, 71-74 (1940), described by Jones et al., Prostaglandins, 24, 279-289 (1982). After completion of all surgical procedures, the lungs were overinflated by two times tidal volume for two breaths by closing the outlet port of the respirator. This was done to standardize the volume history of the lungs. Five minutes after initiation of artificial respiration, when the Pao had stabilized, a succession of doubling concentrations of methacholine from 25 to 1600 μg/ml was given for 20 s at intervals of 5 min using an ultrasonic nebulizer until a 200% increase in Pao was recorded or the dose sequence was completed. The nebulizer generated the aerosol at a rate of 15.2 μl/min during the 20-s period, and 46.4% of the generated aerosol was deposited in the lungs as measured by a radioaerosol technique. See Minami et al., Jpn. J. Chest Dis., 21, 252-258 (1982). The median aerodynamic diameter of the particles of normal saline was 3.59±1.96 μm (mean±SD).

IL-5-Induced Airway Eosinophil Accumulation

Guinea pigs weighing 350 to 400 g were treated i.p. before IL-5 injection with TFMK (20 mg/kg), E6123 (0.1 mg/kg), or vehicle (n=7 in each group). Animals were lightly anesthetized with diethyl ether inhalation, and then 200 μl sterile PBS containing 20 ng IL-5 was intranasally injected into the airway.

Analysis of Bronchoalveolar Cells

After assessment of antigen-induced airway hyperresponsiveness or 24 h after IL-5 injection, airway inflammation was assessed. Immediately before and 1, 24, 48, and 72 h after antigen challenge, bronchoalveolar lavage (BAL) was performed to determine the time-course of cellular infiltration into airways. Two aliquots of 10 ml sterile 37° C. saline were infused into the tracheal cannula. The BAL fluid was recovered manually by gentle aspiration with a disposable syringe after each infusion. The total cell number was determined using a Turk solution. Differential cell counts were performed on cytospin preparations (Cytospin 2, Shandon, England) by counting 300 cells after staining by the May-Grüwald-Giemsa method in a single-blind method.

Lung Histology

After washing with sterile saline, the lung was fixed by perfusion with 10% formalin. Lung sections were stained with hematoxylin and eosin.

Effect of i.p. TFMK on TXB₂ Production ex vivo

Sixty min after TFMK (20 mg/kg or vehicle i.p.; n=4 in each group), guinea pigs were anesthetized and heparinized blood (20 U/ml) was obtained via right carotid artery. Duplicate 1-ml aliquots of whole blood were incubated first with 5 μg/ml CB for 2 min and subsequently with 10⁻⁶ M FMLP for 30 additional min at room temperature. Control samples from the same animals were incubated with buffer alone. Immediately after centrifugation, TXB₂ was measured by enzyme immunoassay using reagents obtained from Cayman Chemical (Ann Arbor, Mich.).

Statistical Analysis

Airway responsiveness to inhaled methacholine was expressed as the dose of methacholine required to provoke a 200% increase (PC₂₀₀) in the Pao. Values for PC200 were logarithmically transformed for analysis and reported as the geometric mean [geometric SEM (GSEM)]. All measurements except for PC200 were expressed as mean±SEM. Variation between three or more groups was analyzed using analysis of variance (ANOVA) followed by Fisher's protected least significant difference. Variation between two groups was tested using Student's t test. A value of p<0.05 was accepted as an indication of statistical significance.

It is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention. Thus, the invention is defined not by the above description, but by the following claims and their equivalents.

What is claimed is:

1. A method for reducing inflammation in a patient, comprising:
administering a compound that reduces an activity of cytosolic phospholipase A₂ (cPLA₂) in combination with a pharmaceutically acceptable carrier.

2. The method according to claim 1, wherein the compound interferes with conversion of membrane phospholipids into lysophospholipid.

3. The method according to claim 1, wherein the compound reduces biosynthesis of inflammatory mediators selected from prostaglandins (PGs), leukotrienes (LTs) and lysophospholipids.

4. The method according to claim 3, wherein lysophospholipids includes platelet activating factor (PAF).

5. The method according to claim 1, wherein the inflammation includes a chronic inflammatory disorder selected from upper or lower airway hyperresponsiveness, asthma, rhinitis, idiopathic pulmonary fibrosis, adult respiratory distress syndrome, pulmonary edema associated with sepsis and gastric acid aspiration, rheumatoid arthritis, and inflammatory bowel disease.

6. A method for reducing migration of white blood cells into tissues, comprising:
reducing adhesion of the white blood cells and diapedesis into tissues by administering a compound that reduces an activity of cytosolic phospholipase A₂ (cPLA₂) in combination with a pharmaceutically acceptable carrier.

7. The method according to claim 6, wherein the white blood cells are selected from granulocytes, eosinophils, basophils, mast cell precursors, macrophages, T-helper cells, T-helper cell precursors, and combinations thereof.

8. A pharmaceutical composition comprising a compound that inhibits the serine-228 residue of cPLA2.

9. The pharmaceutical composition according to claim 8 wherein the compound is selected from trifluoromethylketone (TMFK) and arachanoic acid bromic methylketone (AABrMK).
